(19) United States
(12) Patent Application Publication
(10) Pub. No.: US 2026/0034319 A1
(43) Pub. Date: Feb. 5, 2026

(54) CARTRIDGE AND INHALER INCLUDING THE SAME

(71) Applicant: KT&G CORPORATION, Daejeon (KR)

(72) Inventors: Moon Won KIM, Daejeon (KR); Yong Mi Jung, Daejeon (KR); Jun Won Shin, Daejeon (KR)

(73) Assignee: KT&G CORPORATION, Daejeon (KR)

(21) Appl. No.: 19/282,171

(22) Filed: Jul. 28, 2025

(30) Foreign Application Priority Data
Aug. 1, 2024 (KR) 10-2024-0102700

(51) Int. Cl. A61M 15/00

(52) U.S. Cl. CPC A61M 15/0061; A61M 15/002; A61M 15/0021; A61M 2205/121; A61M 2210/0625

(57) ABSTRACT

A cartridge for an inhaler includes a housing forming an exterior of the cartridge, an accommodation portion formed in the housing to accommodate a target substance, a passage portion formed in the housing to fluidly connect an exterior of the housing to the accommodation portion, and an opening portion formed in the housing to allow the target substance accommodated in the accommodation portion to pass therethrough, wherein the accommodation portion includes a first surface extending in a first direction and a second surface inclined with respect to the first direction, one end of the second surface is connected to the passage portion, and another end of the second surface is connected to the opening portion.

### CROSS-REFERENCE TO RELATED APPLICATION

This application is based on and claims priority under 35 U.S.C. § 119 to Korean Patent Application No. 10-2024-0102700, filed on Aug. 1, 2024, in the Korean Intellectual Property Office, the disclosure of which is incorporated by reference herein in its entirety.

### BACKGROUND

### 1. Field

Various embodiments of the disclosure relate to a cartridge and an inhaler including the cartridge, and more particularly, to a cartridge having an improved internal structure, and an inhaler including the cartridge.

### 2. Description of the Related Art

Studies have been conducted on inhalers that directly deliver target substances to a user's lungs. In this case, a target substance may refer to theanine, caffeine, taurine, nicotine, etc. and may have a fine granule or dry powder form.

The target substance may be stored in a cartridge. In this case, the cartridge may be mounted on an inhaler and used. When the target substance is completely consumed, the target substance may be refilled or the cartridge may be replaced to continue using the inhaler.

Fields to which the inhaler is applied include electronic cigarettes that store tobacco substances, drug inhalation devices that aerosolize medicinal substances for the treatment of diseases such as asthma or pulmonary diseases, and the like.

### SUMMARY

When a user puts the mouthpiece of an inhaler in his/her mouth and inhales a target substance, the target substance moves through a cartridge and the interior of the inhaler together with air and may be inhaled into the user's mouth. In this case, it is necessary to design the cartridge and the inhaler so that air may effectively transport the target substance.

In addition, when designing the inhaler, a hole through which air may flow into the cartridge has to be arranged, but there is a possibility that the target substance may flow out of the inhaler through the hole. Therefore, it is necessary to design the cartridge and the inhaler to reduce this phenomenon.

The target substance stored inside the cartridge may be in a coagulated form by clumping. From the perspective of a user inhaling the target substance, since the smaller the particles, the less discomfort there is for the user to inhale the target substance, a structure that may deagglomerate coagulated particles inside the inhaler before the user inhales the target substance is required.

Embodiments provide a cartridge having an inclined internal structure and an inhaler including the cartridge.

Embodiments also provide a cartridge designed such that an air-inflow portion is bent at least once and an inhaler including the cartridge.

Additionally, embodiments provide a cartridge having a cyclone structure and an inhaler including the cartridge.

The technical problems of the present disclosure are not limited to the above-described description, and other technical problems may be clearly understood by one of ordinary skill in the art from the embodiments to be described hereinafter.

Additional aspects will be set forth in part in the description which follows and, in part, will be apparent from the description, or may be learned by practice of the presented embodiments of the disclosure.

A cartridge for an inhaler according to an embodiment may include a housing forming an exterior of the cartridge, an accommodation portion formed in the housing to accommodate a target substance, a passage portion formed in the housing to fluidly connect an exterior of the housing to the accommodation portion, and an opening portion formed in the housing to allow the target substance accommodated in the accommodation portion to pass through, wherein the accommodation portion may include a first surface extending in a first direction and a second surface inclined with respect to the first direction, one end of the second surface may be connected to the passage portion, and another end of the second surface may be connected to the opening portion.

An inhaler according to an embodiment may include a cartridge including a housing, an accommodation portion formed in the housing to accommodate a target substance, a passage portion formed in the housing to fluidly connect an exterior of the housing to the accommodation portion, and an opening portion formed in the housing to allow the target substance accommodated in the accommodation portion to pass through, and a main body to which the cartridge is detachably coupled, the main body including a body including an accommodation space for accommodating the cartridge, a mouthpiece protruding out of the body and contacting a user's mouth, and a flow path connecting the opening portion of the cartridge to the mouthpiece, wherein the accommodation portion may include a first surface extending in a first direction and a second surface inclined with respect to the first direction, one end of the second surface may be connected to the passage portion, and another end of the second surface may be connected to the opening portion.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view showing a main body of an inhaler according to an embodiment and a cartridge separated from the main body of the inhaler;

FIG. 2A is a perspective view of a cartridge according to an embodiment;

FIG. 2B is a cross-sectional view of the cartridge of FIG. 2A taken along line A-A′ in FIG. 2A;

FIG. 3A is a perspective view of an inhaler main body according to an embodiment;

FIG. 3B is a cross-sectional view of the inhaler main body of FIG. 3A taken along line B-B′ in FIG. 3A;

FIG. 4 is a side view of an inhaler according to an embodiment;

FIG. 5 is a perspective view showing another example of a chamber that may be applied to the inhaler of FIG. 4;

FIG. 6 is a cross-sectional view of a cartridge according to another embodiment to which a door is applied;

FIGS. 7A to 7C are cross-sectional views of cartridges according to other embodiments;

FIG. 8 is a perspective view of a cartridge according to another embodiment to which a blocking portion is applied;

FIGS. 9A and 9B are cross-sectional views of an inhaler according to another embodiment, equipped with a cartridge that may be used upside down;

FIG. 10A is a cross-sectional view of a cartridge according to another embodiment in which a door is applied to the cartridge of FIG. 9A;

FIG. 10B is a cross-sectional view of a cartridge according to another embodiment in which a cartridge blocking portion of FIG. 9A is applied;

FIGS. 11A and 11B are perspective views showing a main body of an inhaler and a cartridge separated therefrom, according to embodiments to which a fastening structure is applied;

FIG. 12 is a perspective view of the main body and cartridge illustrated in FIG. 11B, viewed from a different angle to explain the internal structure of the main body of the inhaler;

FIG. 13A is an exploded front view showing the inhaler main body illustrated in FIG. 12 in a first operating state;

FIG. 13B is an exploded front view showing the inhaler main body illustrated in FIG. 12 in a second operating state;

FIG. 14A is a cut-away side view schematically showing a state in which a fastening groove of a cartridge is coupled to an inhaler main body through fastening with a fastening member in a first operating state; and

FIG. 14B is a cut-away side view schematically showing a state in which a fastening groove of a cartridge is coupled to an inhaler main body through fastening with a fastening member in a second operating state.

### DETAILED DESCRIPTION

Regarding the terms in the various embodiments, the general terms which are currently and widely used are selected in consideration of functions of structural elements in the various embodiments of the present disclosure. However, meanings of the terms can be changed according to intention, a judicial precedence, the appearance of a new technology, and the like. In addition, in certain cases, terms which can be arbitrarily selected by the applicant in particular cases. In such a case, the meaning of the terms will be described in detail at the corresponding portion in the description of the present disclosure. Therefore, the terms used in the various embodiments of the present disclosure should be defined based on the meanings of the terms and the descriptions provided herein.

In addition, unless explicitly described to the contrary, the word “comprise” and variations such as “comprises” or “comprising” will be understood to imply the inclusion of stated elements but not the exclusion of any other elements. In addition, the terms “-er”, “-or”, and “module” described in the specification mean units for processing at least one function and operation and can be implemented by hardware components or software components and combinations thereof.

As used herein, when an expression such as “at least any one” precedes arranged elements, it modifies all elements rather than each arranged element. For example, the expression “at least any one of a, b, and c” should be construed to include a, b, c, or a and b, a and c, b and c, or a, b, and c.

In the description of embodiments of the disclosure, certain detailed explanations of the related art are omitted when it is deemed that they may unnecessarily obscure the essences of embodiments of the disclosure. In addition, the accompanying drawings are only intended to facilitate understanding of the embodiments described herein, and the spirit of the disclosure is not limited by the accompanying drawings and should be understood to include all changes, equivalents or alternatives included in the spirit and scope of the disclosure.

While such terms as “first”, “second”, etc., may be used to describe various components, such components must not be limited to the above terms. The above terms are used only to distinguish one component from another.

When an element is referred to as being “connected to” or “coupled to” another element, it may be directly connected or coupled to the other element or intervening elements may be present. In contrast, when an element is referred to as being “directly connected to” or “directly coupled to” another element, there are no intervening elements present.

An expression used in the singular encompasses the expression of the plural, unless it has a clearly different meaning in the context.

Hereinafter, the present disclosure will now be described more fully with reference to the accompanying drawings, in which exemplary embodiments of the present disclosure are shown such that one of ordinary skill in the art may easily work the present disclosure. The same or similar components will be assigned the same reference numerals regardless of the reference numerals in the drawings, and the same descriptions thereof will be omitted.

The disclosure may, however, be embodied in many different forms and should not be construed as being limited to the embodiments set forth herein.

Hereinafter, embodiments of the disclosure will be described in detail with reference to the drawings.

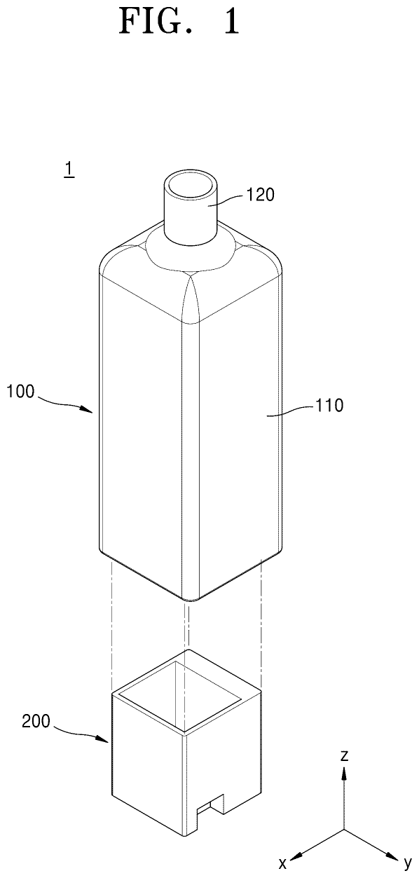

FIG. 1 is a perspective view showing a main body of an inhaler according to an embodiment and a cartridge separated from the main body of the inhaler.

Referring to FIG. 1, an inhaler 1 according to an embodiment may include a main body 100 and a cartridge 200.

The main body 100 may occupy most of the exterior of the inhaler 1. The cartridge 200 may be detachably coupled to a portion of the main body 100 to form a portion of the exterior of the inhaler 1 together with the main body 100.

Embodiment are not limited to an example in which the cartridge 200 is detachably coupled to the main body 100, and the cartridge 200 may be formed integrally with the main body 100. However, the following description will focus on an embodiment in which the cartridge 200 is detachably coupled to the main body 100 and each of the main body 100 and the cartridge 200 is treated as a component of the inhaler 1. In this case, the remaining configuration excluding the cartridge 200 may be referred to as the main body 100.

Referring to FIG. 1, the main body 100 may include a body 110 and a mouthpiece 120.

The body 110 may form the exterior of the inhaler 1 and may perform the function of accommodating and protecting components of the inhaler 1. For example, the body 110 may accommodate the cartridge 200.

As illustrated in the drawings, the body 110 may have a rectangular solid shape, but is not limited thereto and may be manufactured in various shapes, such as a cylindrical shape, an elliptical column, or a polygonal column. That is, embodiments are not limited to the shape of the body 110 illustrated in FIG. 1.

The body 110 may include an accommodation space (not shown) for accommodating the cartridge 200 therein. The accommodation space may be formed in a lower portion of the body 110. The accommodation space may be opened downwardly to allow access of the cartridge 200. At least a portion of the cartridge 200 may be accommodated inside the body 110 through an opening at the lower side of the accommodation space. The accommodation space may have a shape corresponding to the shape of the cartridge 200. For example, the accommodation space may have a rectangular parallelepiped shape.

The mouthpiece 120 is configured to function as a passage for a target substance stored inside the inhaler 1 to move to the outside of the inhaler 1. The mouthpiece 120 may be arranged on an upper portion of the body 110 and may come into contact with the user's mouth. The mouthpiece 120 may be formed to protrude from the body 110 in the +z direction. The mouthpiece 120 may have a shape that may easily come into contact with the user's mouth. The user may inhale the target substance after bringing the mouth into contact with the mouthpiece 120 formed in the body 110.

The cartridge 200 may accommodate the target substance. In this case, the target substance may include one or more of various forms, such as dry powder and fine granule. When the target substance is in the form of dry powder, the cartridge 200 may store powder of about 30 mg, 50 mg, 100 mg, 300 mg, 600 mg, or 1000 mg.

When the target substance is in the form of dry powder, the target substance may be composed of only an inhalable powder of about 1 μm to about 5 μm or about 0.5 μm to about 10 μm. In addition, the target substance may be composed of a mixed form of an inhalable powder of about 1 μm to about 5 μm or about 0.5 μm to about 10 μm and a carrier of about 10 μm to about 20 μm or about 5 μm to about 50 μm.

Powders that may be applied as inhalable materials may include nicotine salts, such as nicotine, nicotine tartrate, nicotine lactate, or nicotine citrate, or may include other functional materials, such as caffeine. In addition, powders that may be applied as inhalable materials may include materials that may bring about other pharmacological effects.

Powders that may be applied as carrier materials may include, for example, powders containing lactose, menthol, or peppermint, or powders in which other essential fragrance oils are powdered.

Materials that may be applied as carriers may include, for example, alpha-lactose monohydrated, beta cyclodextrin, maltodextrin, or mannitol, and may be in a form in which the above excipients are appropriately mixed. In addition, the above excipients and other excipients such as saccharin and xylitol may be included.

The cartridge 200 may be detachably coupled to the body 110 of the main body 100 while containing the target substance therein. For example, the cartridge 200 may be mounted on the main body 100 by inserting at least a part of the cartridge 200 into the main body 100, or by inserting at least a part of the main body 100 into the cartridge 200.

The main body 100 and the cartridge 200 may be maintained in a coupled state by a snap-fit method, a screw coupling method, a magnetic coupling method, a forced fit method, or the like. However, the coupling method of the main body 100 and the cartridge 200 is not limited to the above-described method.

The cartridge 200 may be used as a consumable in the inhaler 1. When the target substance accommodated in the cartridge 200 is exhausted during the use of the inhaler 1, the user may separate the cartridge 200 from the main body 100 and use a new cartridge 200 by coupling the new cartridge 200 to the main body 100.

However, embodiments are not limited to the example in which the cartridge 200 is used as a consumable. According to an embodiment, the cartridge 200 may be used in a manner of refilling the target substance, such as by filling a new target substance into the same cartridge 200.

Although not shown in the drawings, the inhaler 1 according to an embodiment may further include a cap (not shown) that may be mounted on the mouthpiece 120. The user may mount the cap on the mouthpiece 120 when the inhaler 1 is not in use.

The cap may prevent the target substance inside the inhaler 1 from leaking out through the mouthpiece 120. In addition, the cap may prevent foreign materials from entering the interior of the inhaler 1 through the mouthpiece 120.

Hereinafter, the internal structure of the inhaler 1 and the movement path of the target substance will be described in detail.

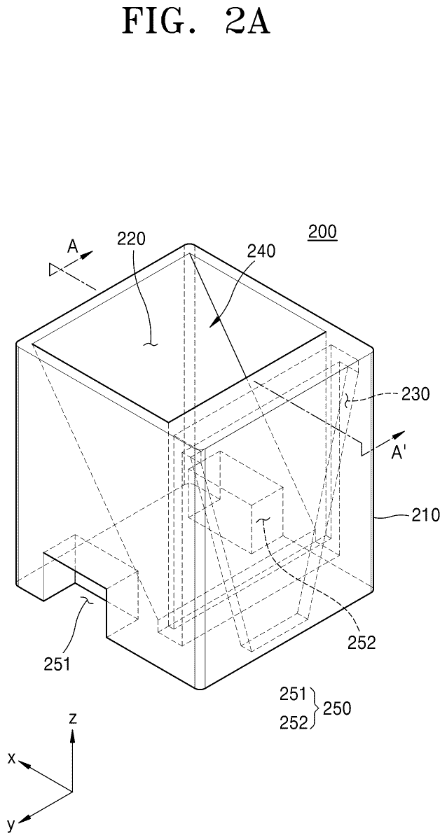

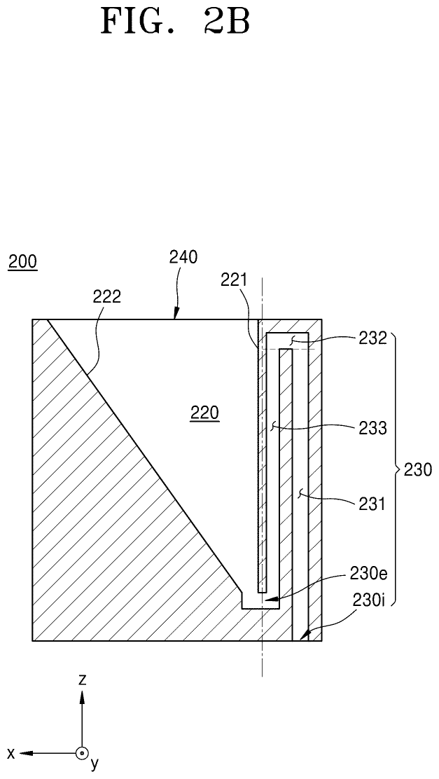

FIG. 2A is a perspective view of a cartridge according to an embodiment. FIG. 2B is a cross-sectional view of the cartridge taken along line A-A′ in FIG. 2A.

Referring to FIGS. 2A and 2B, a cartridge 200 according to an embodiment may include a housing 210, an accommodation portion 220, a passage portion 230, an opening portion 240, and a groove portion 250.

The housing 210 is a component that forms the exterior of the cartridge 200. In this case, the accommodation portion 220, the passage portion 230, the opening portion 240, and the groove portion 250 of the cartridge 200 may be components formed in the housing 210. That is, the listed components may be components included in the cartridge 200 and may be components included in the housing 210. The components of the cartridge 200 may be determined according to the shape of the housing 210.

The accommodation portion 220 is a component formed in the housing 210 to accommodate the target substance. In this case, the accommodation portion 220 may correspond to a groove formed in a part of the housing 210. The target substance may be stored in the accommodation portion 220.

The accommodation portion 220 may include a first surface 221 extending in a first direction (e.g., in the z-axis direction) and a second surface 222 inclined with respect to the first direction. The target substance may be located between the first surface 221 and the second surface 222.

One end of the second surface 222 may be connected to the passage portion 230 to be described later, and the other end of the second surface 222 may be connected to the opening portion 240 to be described later. Air introduced into the accommodation portion 220 may move along the second surface 222 and transport the target substance from the passage portion 230 toward the opening portion 240. In this case, because the second surface 222 has an inclined structure, a small amount of the target substance remaining inside the accommodation portion 220 may also move along the inclined surface together with the air.

The passage portion 230 is configured to fluidly connect the outside of the housing 210 to the accommodation portion 220. In this case, ‘fluid connection’ may mean that elements are connected so that a fluid such as air may pass through and flow. Hereinafter, the expression ‘connection’ may include the meaning of fluid connection.

The passage portion 230 may correspond to a passage formed in a part of the housing 210. The passage portion 230 may function as a passage through which air flows into the interior of an inhaler when a user uses the inhaler. That is, the passage portion 230 may correspond to a component through which air passes first when air flows into the interior of the inhaler or the cartridge 200. In this case, the size of the passage portion 230 may be sufficiently small to provide sufficient suction resistance to the user of the inhaler 1.

The target substance stored in the accommodation portion 220 may flow out of the housing 210 or the cartridge 200 through the passage portion 230. To solve this problem, the passage portion 230 may include a shape bent at least once.

Hereinafter, the shape of the passage portion 230 will be specifically described. The passage portion 230 may include an air-introducing inlet 230i, a first passage 231, a second passage 232, a third passage 233, and an air inlet 230e.

The air-introducing inlet 230i is configured to be opened toward the outside of the housing 210 and connected to the outside of the housing 210. The air-introducing inlet 230i may function as an inlet of the passage portion 230.

The air-introducing inlet 230i may be opened toward a lower part (e.g., in the z-axis direction) of the cartridge 200. Because the cartridge 200 approaches and connects to an inhaler main body (e.g., the main body 100 of FIG. 1) in the z-axis direction, even when there is no separate opening in the main body, the air-introducing inlet 230i may function as an inlet of the inhaler.

However, depending on embodiments, a direction in which the air-introducing inlet 230i is opened may vary, and accordingly, an opening connected to the air-introducing inlet 230i of the cartridge 200 may be arranged in the main body. Also, even when the cartridge 200 approaches and connects to the main body in a different direction, the air-introducing inlet 230i and the opening of the main body connected thereto may be positioned at an appropriate locations.

The first passage 231 is a passage extending from the air-introducing inlet 230i in the first direction (e.g., in the z-axis direction). Referring to the drawings, the air-introducing inlet 230i is opened in the first direction and the first passage 231 extends in a direction in which the air-introducing inlet 230i is opened, but embodiments are not limited thereto. The direction in which the air-introducing inlet 230i is opened and the direction in which the first passage 231 extends may vary depending on embodiments.

The second passage 232 is a passage connected to the first passage 231 and extending in a second direction (e.g., in the x-axis direction) that crosses the first direction. In this case, the second passage 232 may connect the first passage 231 to the third passage 233. Referring to the drawings, the second passage 232 is formed shorter than the first passage 231 or the third passage 233, but embodiments are not limited thereto. The ratio of the length of the first passage 231 or the third passage 233 to the length of the second passage 232 may vary depending on embodiments.

The third passage 233 is a passage that is connected to the second passage 232 and extends in the first direction. That is, the third passage 233 may extend in the same direction as the first passage 231. However, embodiments are not limited to the same direction. The direction in which the first passage 231 and the third passage 233 extend may vary depending on embodiment

The air inlet 230e is configured to be opened toward the accommodation portion 220 and connected to the accommodation portion 220. The air inlet 230e is connected to the third passage 233 and may function as an outlet of the passage portion 230. The air inlet 230e may be formed on the first surface 221 of the accommodation portion 220.

Referring to the drawings, the air inlet 230e is opened in the second direction and the third passage 233 extends in a direction crossing the direction in which the air inlet 230e is opened, but embodiments are not limited thereto. The direction in which the air-introducing inlet 230i is opened and the direction in which the first passage 231 extends may vary depending on embodiments, and the two directions may be the same.

In summary, air may reach the accommodation portion 220 from the outside of the housing 210 through the air-introducing inlet 230i, the first passage 231, the second passage 232, the third passage 233, and the air inlet 230e.

Because the target substance is located between the first surface 221 and the second surface 222 of the accommodation portion 220, the target substance may be accumulated in the accommodation portion 220 in the first direction (e.g., the z-axis direction). In this case, the accommodation portion 220 and the passage portion 230 may be arranged in parallel in the second direction (e.g., the x-axis direction) that crosses the first surface 221 of the accommodation portion 220. The air inlet 230e connected to the accommodation portion 220 may be opened in the second direction that crosses the first direction.

Accordingly, when the cartridge 200 is arranged so that the first direction is the direction of gravity, the target substance stored in the accommodation portion 220 may be prevented from flowing out through the air inlet 230e.

In addition, as shown in the drawings, a part where the air inlet 230e and the third passage 233 are connected to each other, a part where the third passage 233 and the second passage 232 are connected to each other, and a part where the second passage 232 and the first passage 231 are connected to each other are bent. That is, the directions in which the air inlet 230e and the third passage 233 are opened are different from each other, the directions in which the third passage 233 and the second passage 232 are opened are different from each other, and the directions in which the second passage 232 and the first passage 231 are opened are different from each other.

Accordingly, even when the target substance stored in the accommodation portion 220 flows back to the passage portion 230, the phenomenon of the target substance flowing out of the housing 210 along the passage portion 230 may be prevented due to a curved shape of the passage portion 230.

In addition, the first passage 231 and the third passage 233 may be arranged parallel in the second direction. Specifically, when the air flowing into the cartridge 200 moves along the first passage 231, the air may move in the +z direction, and when the air moves along the third passage 233, the air may move in the −z direction.

On the other hand, the target substance flowing back from the accommodation portion 220 has to move in the +z direction when moving along the third passage 233, and in the −z direction when moving along the first passage 231 in order to flow out of the housing 210.

Accordingly, because the target substance has to move in various directions to flow out of the housing 210 or the cartridge 200 through the passage portion 230, the phenomenon of the target substance flowing out of the housing 210 along the passage portion 230 may be prevented.

The cross-sectional area of a cross-section of the first passage 231 cut in the second direction may increase away from the air-introducing inlet 230i. By adopting this structure, air may flow into the relatively narrow air-introducing inlet 230i and move along the first passage 231 to gradually spread to a wider area.

When the cartridge 200 is designed to have a consistently large width from the air-introducing inlet 230i to the end of the first passage 231, even when the user tries to inhale only a small amount of the target substance, a large amount of the target substance may be inhaled by the user. In addition, the suction resistance and the pressure applied to the passage portion 230 may be low, which may cause false suction. According to an embodiment, these phenomena may be prevented.

The opening portion 240 is a component formed in the housing 210 so that the target substance accommodated in the accommodation portion 220 may pass through when moving together with air. Specifically, air introduced into the accommodation portion 220 through the passage portion 230 may transport the target substance. The air together with the target substance may pass through the opening portion 240 and move to the outside of the housing 210 or the cartridge 200. That is, the opening portion 240 may correspond to a component through which air passes through the interior of the cartridge 200 for the last time. In addition, when the target substance is completely consumed, the user may refill the target substance into the accommodation portion 220 through the opening portion 240.

Referring to the drawings, one end of the first surface 221 and one end of the second surface 222 may be connected to the air inlet 230e of the passage portion 230, and the other end of the first surface 221 and the other end of the second surface 222 may be connected to the opening portion 240.

Because the second surface 222 is an inclined surface inclined with respect to the first surface 221, one end of the first surface 221 and one end of the second surface 222 may be relatively close to each other, and the other end of the first surface 221 and the other end of the second surface 222 may be relatively far from each other.

As illustrated in the drawings, the space between the other end of the first surface 221 and the other end of the second surface 222 may correspond to the opening portion 240. That is, the open area of the opening portion 240 is relatively large compared to the open area of the air inlet 230e. However, embodiments are not limited thereto. According to an embodiment, an upper part of the accommodation portion 220 may be blocked by the housing 210, and a relatively small opening portion may be arranged in the housing 210.

The opening portion 240 may be opened in the first direction, and the air inlet 230e of the passage portion 230 may be opened in the second direction (e.g., the x-axis direction) that crosses the first direction. When the cartridge 200 is mounted on the inhaler main body, the opening portion 240 may face the inhaler main body. In this case, because the air inlet 230e is opened in a direction that crosses a length direction of the inhaler main body rather than in a direction (e.g., the −z direction) away from the inhaler main body, unwanted leakage of the target substance through the air inlet 230e may be prevented while the user uses the inhaler.

The groove portion 250 is a component in which one edge of the outer side of the housing 210 and one surface of the housing 210 including the one edge are sunken. Two groove portions 250 may be arranged. For example, the groove portion 250 may include a first groove portion 251 and a second groove portion 252 arranged to face each other. The first groove portion 251 may be arranged on one surface of the housing 210, and the second groove portion 252 may be arranged on the other surface of the housing 210 opposite the one surface.

When the cartridge 200 is mounted on the inhaler main body, if the cartridge 200 is completely inserted into the inhaler main body, it may be difficult to separate the cartridge 200 from the inhaler main body. In this case, because the groove portion 250 is arranged on the cartridge 200, the user may hold the first groove portion 251 and the second groove portion 252 with his or her hand and separate the cartridge 200 from the inhaler main body.

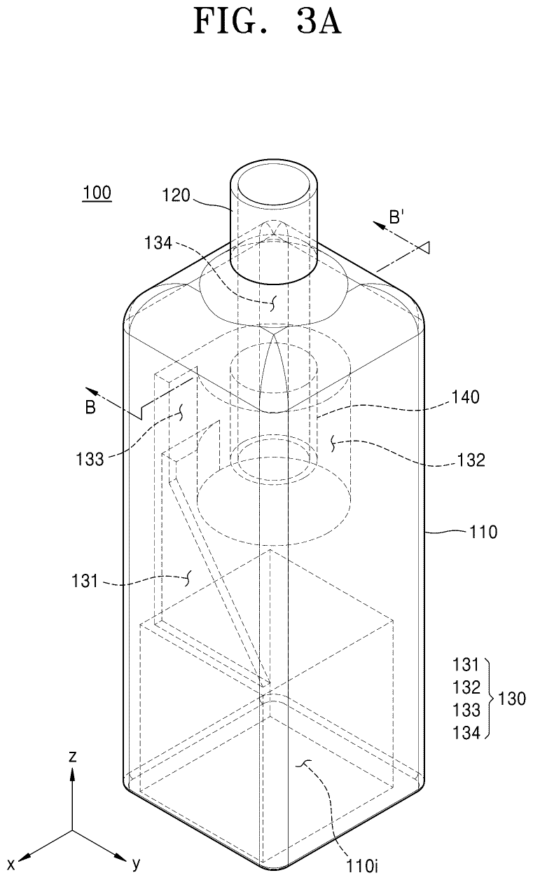

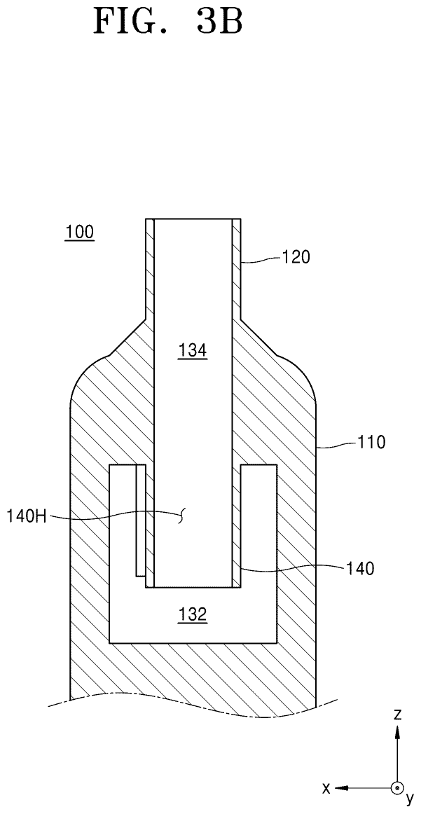

FIG. 3A is a perspective view of an inhaler main body according to an embodiment. FIG. 3B is a cross-sectional view of the inhaler main body taken along line B-B′ in FIG. 3A.

Referring to FIGS. 3A and 3B, an inhaler main body 100 according to an embodiment may include a body 110, a mouthpiece 120, and a flow path 130.

At least one of the components of the inhaler illustrated in FIGS. 3A and 3B may be identical or similar to at least one of the components of the inhaler 1 illustrated in FIG. 1, and any duplicate description is omitted below.

The flow path 130 is a component that connects a cartridge (e.g., the cartridge 200 of FIG. 1) to the mouthpiece 120. The flow path 130 may be fluidly connected to an opening portion (e.g., the opening portion 240 of FIG. 2A) of the cartridge 200. A target substance stored in the cartridge 200 may pass through the opening portion and flow into the flow path 130. Thereafter, the target substance may move along the flow path 130 and be inhaled by the user.

The main body 100 may further include a protrusion 140 for efficient transfer of the target substance. The protrusion 140 includes a hollow 140H connected to the mouthpiece 120 and is configured to protrude inwardly from the body 110. While the mouthpiece 120 protrudes outwardly from a part of the body 110, the protrusion 140 may protrude inwardly from a part of the body 110. The mouthpiece 120 and the protrusion 140 may be aligned in a row. A target substance moving along the hollow 140H of the protrusion 140 may reach the mouthpiece 120 and be inhaled by the user.

Hereinafter, the flow path 130 will be described in detail. The flow path 130 may include an inlet portion 131, a chamber 132, a connection portion 133, and an outlet portion 134.

The inlet portion 131 is configured to be opened toward an accommodation space 110i of the body 110 and connected to the cartridge. The inlet portion 131 may function as an inlet of the flow path 130.

The inlet portion 131 may be opened toward a lower part (e.g., in the z-axis direction) of the body 110. When the cartridge approaches the inhaler main body (e.g., the main body 100 of FIG. 1) in the z-axis direction and is coupled to the accommodation space 110i, the positions of the inlet portion 131 and the opening portion of the cartridge may be aligned. Accordingly, air passing through the cartridge 200 may be introduced into the inlet portion 131 through the opening portion.

As illustrated in the drawings, the inlet portion 131 is opened in the first direction (e.g., in the z-axis direction) together with the opening portion, but the direction in which the inlet portion 131 is opened may vary depending on embodiments. In this case, the position of the opening portion of the cartridge may also vary depending on the position of the inlet portion 131. The positions of the inlet portion 131 and the opening portion may be determined so that air passing through the opening portion may flow into the inlet portion 131.

The chamber 132 is an empty space having a cylindrical shape. The chamber 132 is formed inside the body 110, and a wall constituting the chamber 132 may correspond to a part of the body 110. The air flowing into the chamber 132 through the inlet portion 131 may rotate along the chamber 132 having a cylindrical shape. The chamber 132 may be arranged below the mouthpiece 120 and connected to the mouthpiece 120. The air rotating along the chamber 132 may move to the mouthpiece 120.

When the protrusion 140 is arranged, the chamber 132 may accommodate the protrusion 140. The air inside the chamber 132 may move to a lower part of the chamber 132 while rotating and be introduced into the hollow 140H of the protrusion 140. The air introduced into the hollow 140H may move to the mouthpiece 120.

A structure designed to allow air to rotate along a wall surface of the chamber 132 and move downward may be called a cyclone structure. By using the cyclone structure, sufficient eddies may be generated within the chamber 132, and when a strong flow rate is added to the eddies, target substances that have clumped together may be de-agglomerated.

In addition, relatively large particles or foreign materials may be dropped to the lower part of the chamber 132, and only relatively small particles may be introduced into the hollow 140H through the air. Accordingly, particles larger than a certain size may be prevented from entering the user's mouth, thereby improving the inhalation sensation of the target substance.

The connection portion 133 is a passage connecting the inlet portion 131 to the chamber 132. In order to connect the inlet portion 131 extending in the first direction to the chamber 132 arranged in the center portion inside the body 110, the connection portion 133 may extend in the second direction (e.g., the x-axis direction) crossing the first direction.

The connection portion 133 may be connected to the chamber 132 by extending from the end of the inlet portion 131 in a tangential direction of the chamber 132 having a cylindrical shape. When air is introduced into the chamber 132 along the connection portion 133, because the air is introduced in a tangential direction of the cylindrical shape, the air may smoothly rotate along the wall surface of the chamber 132.

The outlet portion 134 is a passage extending from the chamber 132 to the mouthpiece 120. When the protrusion 140 is arranged, the outlet portion 134 may extend along the hollow 140H of the protrusion 140 to the mouthpiece 120. Air passing through the cyclone structure may pass through the outlet portion 134 together with the target substance and be inhaled by the user.

In summary, air may pass through the cartridge 200, pass through the inlet portion 131, the connection portion 133, the chamber 132, and the outlet portion 134, and reach the user's mouth.

The area of a cross-section of the inlet portion 131, in which the cross-section is taken in a direction transverse to a direction in which the inlet portion 131 extends, may decrease toward the connection portion 133. Referring to the drawings, one end of the inlet portion 131 facing the accommodation space 110i is formed wide, while the other end of the inlet portion 131 is formed narrow.

The connection portion 133 connected to the other end of the inlet portion 131 may also extend in the second direction while maintaining a narrow width. As the width of the connection portion 133 becomes narrower, air may be introduced into the chamber 132 closer to the tangential direction of the cylindrical shape. As a result, the air may smoothly rotate along the wall surface of the chamber 132.

Although not shown in the drawings, the inhaler main body 100 may include a mesh net (not shown) arranged in an area inside the mouthpiece 120. The presence of the mesh net may provide suction resistance to the user of the inhaler. In addition, due to the arrangement of the mesh net, the powder for inhalation and the carrier material may collide with the mesh net and be separated from each other and then inhaled by the user.

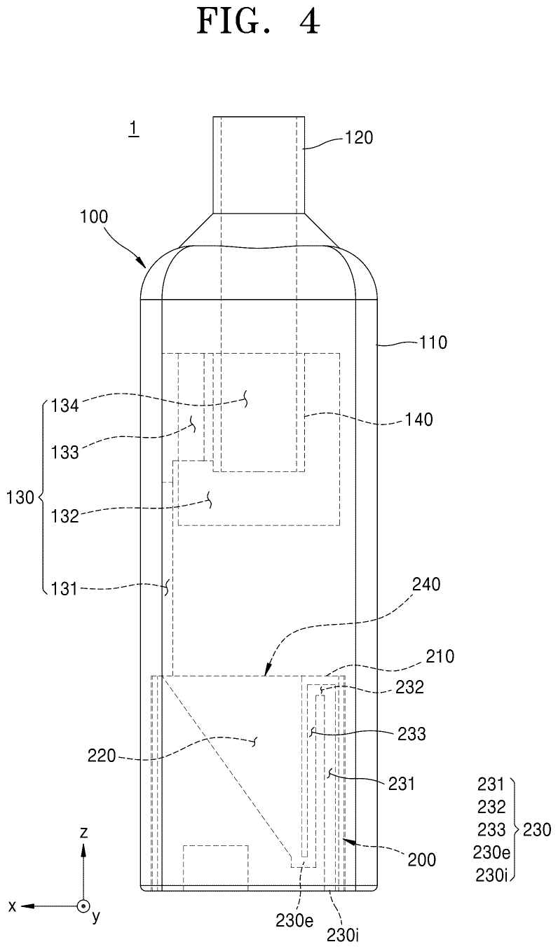

FIG. 4 is a side view of an inhaler according to an embodiment in which an inhaler main body and a cartridge are coupled to each other.

Referring to FIG. 4, an inhaler 1 according to an embodiment may include a main body 100 and a cartridge 200. At least one of the components of the inhaler 1 illustrated in FIG. 4 may be identical or similar to at least one of the components of the inhaler 1 described above, and any duplicate description is omitted below.

When a user of the inhaler 1 puts the mouthpiece 120 in his/her mouth and inhales air or a target substance, air may be introduced into the interior of the inhaler 1 through the air-introducing inlet 230i of the cartridge 200.

The air may sequentially pass through the first passage 231, the second passage 232, and the third passage 233 of the passage portion 230 and be introduced into the accommodation portion 220 through the air inlet 230e. The air may transport a target substance stored in the accommodation portion 220. The air may move along an inclined surface of the accommodation portion 220 together with the target substance and move to the outside of the cartridge 200 through an opening portion 240.

Because the opening portion 240 is connected to the inlet portion 131 of the flow path 130, when the air passes through the opening portion 240, the air may be introduced into the inlet portion 131 of the main body 100. The air introduced into the interior of the body 110 may sequentially pass through the inlet portion 131, the connection portion 133, the chamber 132, and the outlet portion 134 of the flow path 130 and be introduced into the user's mouth through the mouthpiece 120.

In this case, the air passing through the chamber 132 may rotate along the wall surface of the chamber 132. The air rotating in an area adjacent to the outlet portion 134 may be introduced into the outlet portion 134. When the protrusion 140 is arranged, the air passing through the chamber 132 does not simply flow across the chamber 132 into a hollow formed in the protrusion 140, but may rotate along the wall surface of the chamber 132 and may move to a lower part of the chamber 132 and flow into the hollow of the protrusion 140.

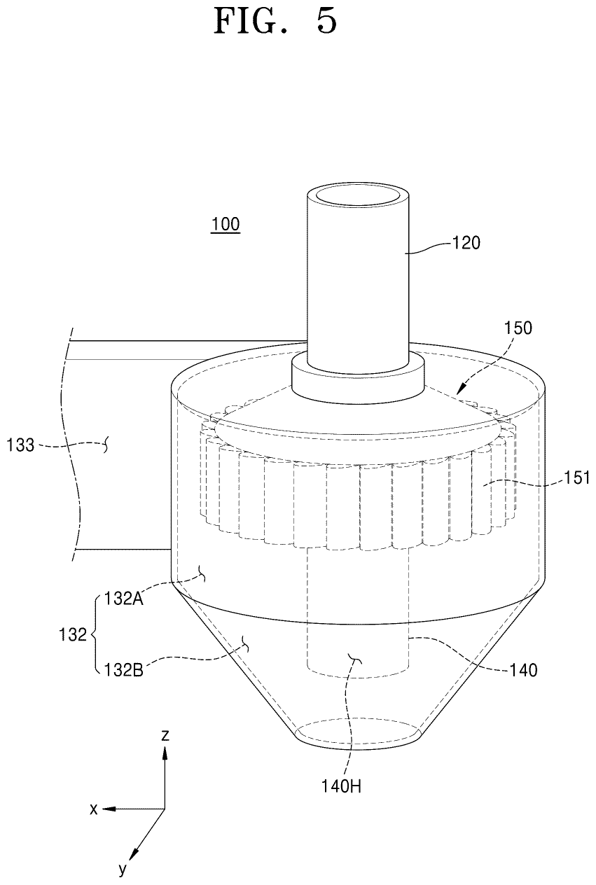

Hereinafter, with reference to FIG. 5, another example in which a cyclone structure is implemented inside the chamber 132 will be described.

FIG. 5 is a perspective view showing another example of a chamber that may be applied to the inhaler of FIG. 4.

Referring to FIG. 5, only a part of the inhaler main body (i.e., the main body) 100 is illustrated. At least one of the components of the main body 100 illustrated in FIG. 5 may be identical or similar to at least one of the components of the main body 100 described above, and any duplicate description is omitted below.

The main body 100 of the inhaler according to another embodiment may further include a guide portion 150. The guide portion 150 is configured to be coupled to the outer surface of the protrusion 140. The guide portion 150 may guide the movement of air so that the air may rotate smoothly inside the chamber 132.

When air is introduced into the chamber 132, because the guide portion 150 is arranged at the center of the chamber 132, the air may only move through the periphery of the chamber 132. That is, the air may move along the wall surface at a position adjacent to the wall surface forming the chamber 132. Accordingly, the air may move while rotating inside the chamber 132.

In addition, the guide portion 150 may include a plurality of wings 151 that guide the movement of the air. Referring to the drawings, the plurality of wings 151 may be arranged to wrap around the guide portion 150 in a clockwise direction when the mouthpiece 120 or the protrusion 140 is used as a rotation axis. Accordingly, the plurality of wings 151 of the guide portion 150 may guide the movement of air so that the air rotates clockwise inside the chamber 132.

However, because a direction in which the air rotates is determined by the position of the connection portion 133 to the chamber 132, a direction in which the wings 151 wrap around the guide portion 150 may vary depending on embodiments.

In the main body 100 of the inhaler according to another embodiment, the chamber 132 may be divided into two regions. Specifically, the chamber 132 may include a first region 132A having a cylindrical shape in which the guide portion 150 is arranged, and a second region 132B in which the end of the protrusion 140 is arranged.

The first region 132A is arranged above the second region 132B, and air introduced into the chamber 132 may sequentially pass through the first region 132A and the second region 132B and then be introduced into the hollow 140H of the protrusion 140. In this case, while the first region 132A may have a cylindrical shape, the second region 132B may have a funnel shape. In other words, the second region 132B may become narrower away from the first region 132A.

According to this shape, as the air rotates in the second region 132B and moves downward, the air may gather in the center of the second region 132B. The air gathered in the center may smoothly move to the hollow 140H of the protrusion 140, and particles falling to the bottom surface of the second region 132B may also gather in a relatively narrow space.

Although not shown in the drawings, the bottom surface of the chamber 132 or the bottom surface of the second region 132B may be opened and closed according to the user's operation. When at least one region of the bottom surface is opened, particles that fall to the bottom surface of the chamber 132 may be discharged through the open region into an accommodation space (e.g., the accommodation space 110i of FIG. 3A).

When a cartridge is mounted in the accommodation space, particles that pass through the open region of the bottom surface of the chamber 132 may pass through an opening portion of the cartridge and be introduced into an accommodation portion. According to this structure, particles introduced into the accommodation portion may move back to the chamber 132 by the user's inhalation, and may undergo deagglomeration by the cyclone structure and be inhaled by the user or settle back to the bottom surface of the chamber 132. When this process is repeated, the target substance filled in the cartridge may be inhaled to the user to the maximum extent.

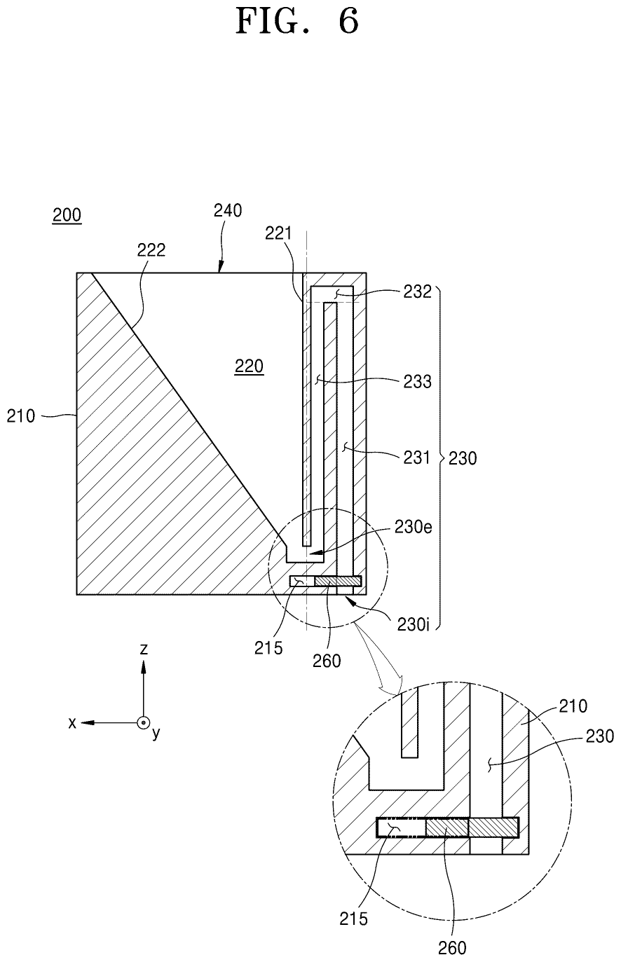

FIG. 6 is a cross-sectional view of a cartridge according to another embodiment to which a door is applied.

Referring to FIG. 6, a cartridge 200 according to another embodiment may further include a door 260 compared to the cartridge 200 of FIG. 2B.

At least one of the components of the cartridge 200 illustrated in FIG. 6 may be identical or similar to at least one of the components of the cartridge 200 described above, and any duplicate description is omitted below.

Despite the curved shape of the passage portion 230, a target substance may move along the passage portion 230 and flow out of the housing 210. In addition, external foreign materials may enter the interior of the cartridge 200 through the passage portion 230. In order to prevent this phenomenon, a door 260 may be placed to block the passage portion 230 when the user is not using the inhaler.

The door 260 is configured to slide to open and close the air-introducing inlet 230i of the passage portion 230. The door 260 may move between an open position and a closed position of the air-introducing inlet 230i.

At least a part of the door 260 may be accommodated in a sliding groove 215 formed in the housing 210. The sliding groove 215 may be arranged adjacent to the air-introducing inlet 230i and may be connected to the air-introducing inlet 230i. In this case, the sliding groove 215 may be formed in the housing 210 to cross the first passage 231.

The door 260 may slide inside the sliding groove 215. That is, the sliding groove 215 may guide the sliding movement of the door 260. Accordingly, the door 260 may only slide in a direction (e.g., in the x-axis direction) in which the sliding groove 215 extends.

Referring to the drawings, the door 260 is in a position to close the air-introducing inlet 230i. In this case, when the door 260 moves along the sliding groove 215, the air-introducing inlet 230i may be opened. The door 260 in the open position is illustrated in a dotted line in the enlarged view.

Although not shown in the drawings, a fastening structure such as a hook may be arranged at one end of the door 260 so that the door 260 does not move in the closed position. Correspondingly, a hole into which a hook is inserted may be arranged in the sliding groove 215. Accordingly, the door 260 may be fixed in the closed position.

In addition, a handle (not shown) protruding outward from the housing 210 may be arranged on the door 260 so that the use may move the door 260 accommodated in the sliding groove 215.

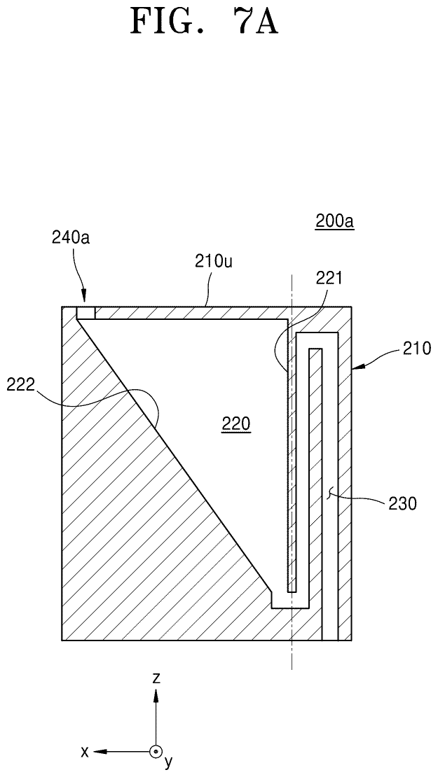

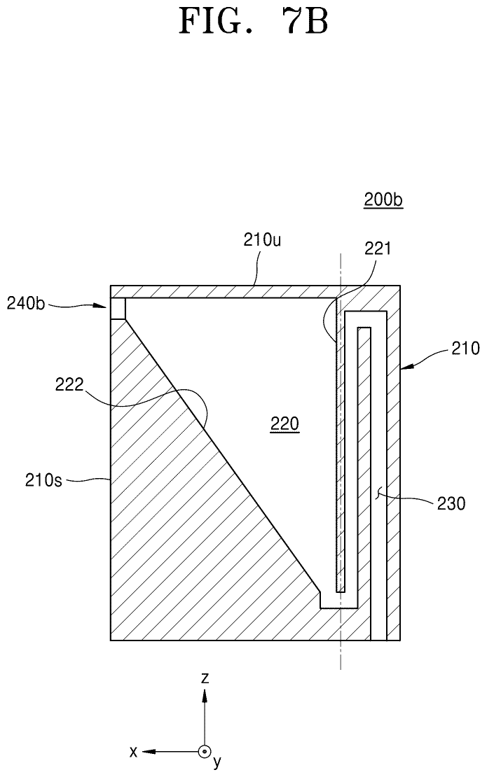

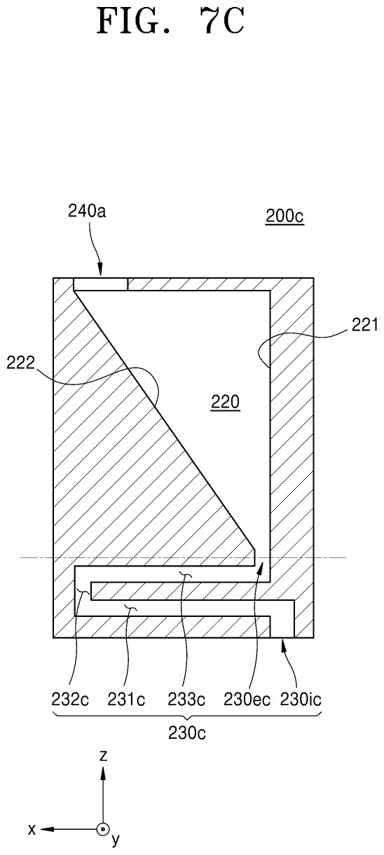

FIGS. 7A to 7C are cross-sectional views of cartridges according to other embodiments.

Referring to FIGS. 7A to 7C, cartridges 200a, 200b, and 200c of various shapes are illustrated, respectively.

At least one of the components of the cartridges 200a, 200b, and 200c illustrated in FIGS. 7A to 7C may be identical or similar to at least one of the components of the cartridge 200 illustrated in FIG. 2B, and any duplicate description is omitted below. In addition, when describing FIGS. 7A to 7C, reference will be made to the drawing symbols of FIG. 4.

Referring to FIG. 7A, the cartridge 200a illustrated in FIG. 7A may further include an upper wall 210u compared to the cartridge 200 of FIG. 2B. In this case, the upper wall 210u may correspond to a component included in the housing 210 or a part of the housing 210.

An opening portion 240a may be arranged in an area of the upper wall 210u. Compared to the cartridge 200 of FIG. 2B, the opening portion 240a may be formed relatively small. For example, the opening portion 240a may have a size corresponding to the inlet of the inlet portion 131 arranged in the main body 100.

Referring to FIG. 7B, in the cartridge 200b illustrated in FIG. 7B, compared to the cartridge 200a of FIG. 7A, an opening area 240b is arranged in a side wall 210s rather than the upper wall 210u. In this case, the side wall 210s may correspond to a component included in the housing 210 or a part of the housing 210.

That is, the opening portion 240b may be arranged to face the first surface 221 of the accommodation portion 220. In order to respond to the position of the opening portion 240b, the inlet of the inlet portion 131 may be formed at a position corresponding to the opening portion 240b in the main body 100 of the inhaler.

Referring to FIG. 7C, in the cartridge 200c illustrated in FIG. 7C, compared to the cartridge 200a of FIG. 7A, a passage portion 230c is arranged at the bottom, not the side, of the accommodation portion 220.

Accordingly, a direction in which each part of the passage portion 230c is opened or extends may also vary. For example, an air-introducing inlet 230ic and an air inlet 230ec may be opened in the first direction (e.g., the z-axis direction), a first passage 231c and a third passage 233c may extend in the second direction (e.g., the x-axis direction), and a second passage 232c connecting two passages, i.e., the first and third passages 231c and 233c, may extend in the first direction.

Up to now, the first direction in which the first surface 221 of the accommodation portion 220 extends has been described as an insertion direction of the cartridge 200 with respect to the main body 100, but according to an embodiment, the second direction crossing the first direction may be the insertion direction of the cartridge 200. In this case, the main body 100 is open toward a lower part of the body 110, and the concept of the cartridge 200 being coupled through an open lower part of the body 110 is the same.

When explaining the cartridge 200a illustrated in FIG. 7A as an example, if the cartridge 200 is coupled through an open lower part of the main body 100 with the second direction as the insertion direction of the cartridge 200, the passage portion 230 may be arranged on an upper part of the accommodation portion 220, and the opening portion 240a may be arranged on a lower side of the accommodation portion 220. In order to respond to the positions of the passage portion 230 and the opening portion 240a, a hole through which air enters and the inlet portion 131 of the flow path 130 may also be arranged in the main body 100.

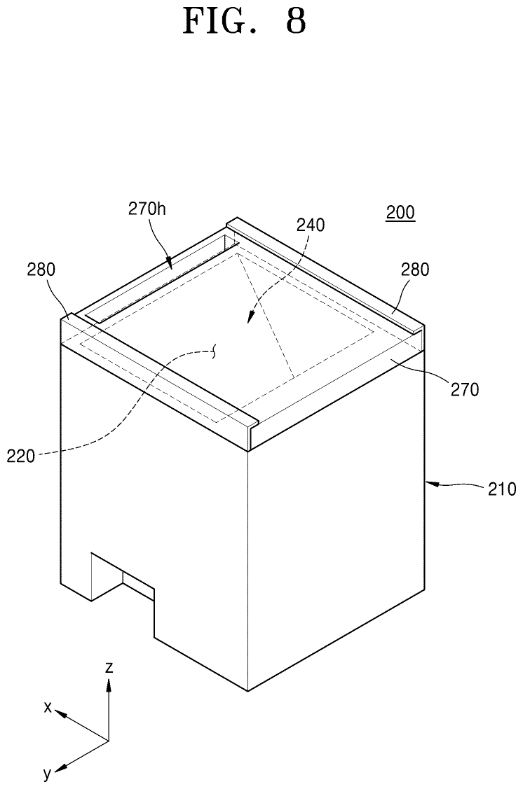

FIG. 8 is a perspective view of a cartridge according to another embodiment to which a blocking portion is applied.

Referring to FIG. 8, a cartridge 200 according to another embodiment may further include a blocking portion 270 and a rail portion 280 compared to the cartridge 200 of FIG. 2A. Hereinafter, when describing FIG. 8, reference will be made to the drawing symbols of FIG. 4.

The blocking portion 270 is configured to cover the opening portion 240. Because the blocking portion 270 blocks at least a part of the opening portion 240, the accommodation portion 220 may be blocked from the outside of the housing 210.

The blocking portion 270 may be detachably coupled to the housing 210. To this end, the rail portion 280 may be arranged on an upper portion of the housing 210. The rail portion 280 may be a component included in the housing 210 or a component independent from the housing 210.

A pair of rail portions 280 may be arranged and may have a shape in which the letter ‘L’ is flipped upside down. The blocking portion 270 may be inserted by sliding between the pair of rail portions 280. In this way, the blocking portion 270 may be coupled to the rail portion 280 and may also be separated from the rail portion 280.

However, a method in which the blocking portion 270 is coupled to the housing 210 is not limited to the method using the rail portion 280. Depending on embodiments, the blocking portion 270 may be detachably coupled to the housing 210 in various ways.

Instead of the blocking portion 270 blocking the opening portion 240, the blocking portion 270 may include an air outlet 270h that fluidly connects the outside of the housing 210 to the accommodation portion 220. The air outlet 270h may be connected to the inlet portion 131 of the main body 100. That is, the air outlet 270h formed in the blocking portion 270 may correspond to an outlet of the cartridge 200 through which air passes while carrying a target substance.

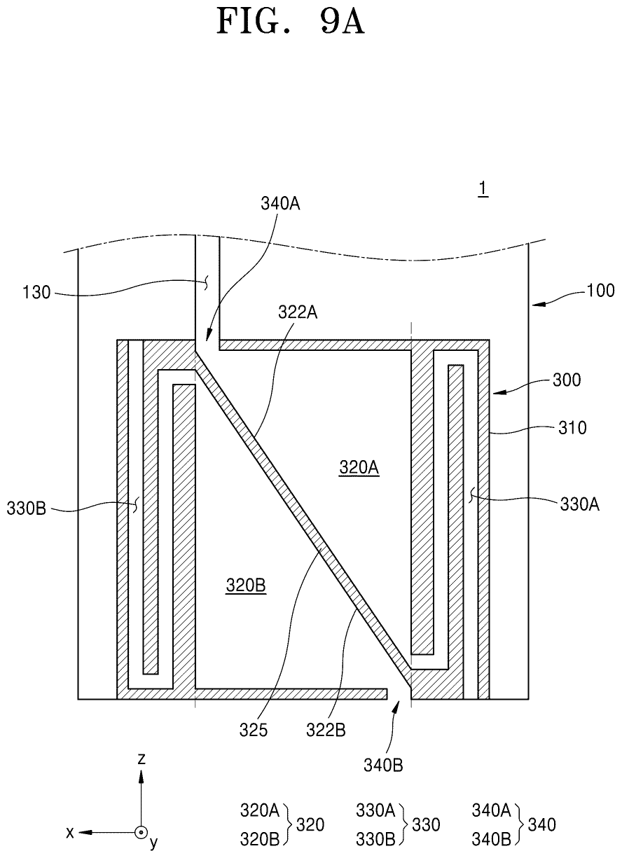

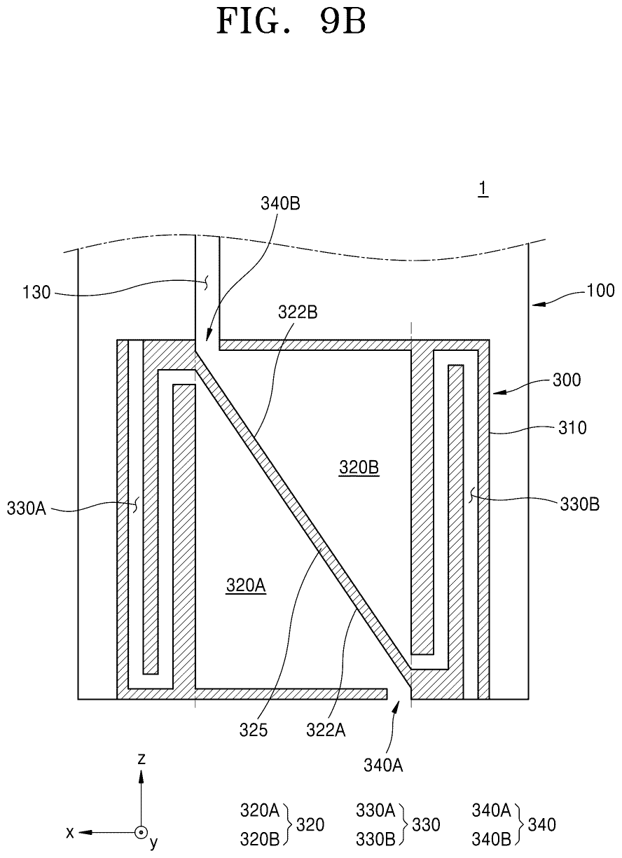

FIGS. 9A and 9B are cross-sectional views of an inhaler according to another embodiment, the inhaler being equipped with a cartridge that may be used upside down.

Referring to FIGS. 9A and 9B, an inhaler 1 according to another embodiment may include a main body 100 and a cartridge 300. At least one of the components of the inhaler 1 illustrated in FIGS. 9A and 9B may be identical or similar to at least one of the components of the inhaler 1 illustrated in FIG. 4, and any duplicate description is omitted below.

The cartridge 300 illustrated in FIGS. 9A and 9B may be used upside down, compared to the cartridge 200 illustrated in FIG. 2B. That is, the cartridge 300 may be mounted on the main body 100 in a direction illustrated in FIG. 9A, and may be mounted on the main body 100 in a direction illustrated in FIG. 9B.

Specifically, regarding the cartridge 300 that may be used upside down, the cartridge 300 may include an accommodation portion 320, a passage portion 330, and an opening portion 340 in a housing 310. The accommodation portion 320 may include two accommodation portions, the passage portion 330 may include two passage portions, and the opening portion 340 may include two opening portions.

The accommodation portion 320 may include a first accommodation portion 320A and a second accommodation portion 320B. The passage portion 330 may include a first passage portion 330A and a second passage portion 330B. The opening portion 340 may include a first opening portion 340A and a second opening portion 340B.

As described above, the first passage portion 330A, the first accommodation portion 320A, and the first opening portion 340A may be fluidly connected to each other, and the second passage portion 330B, the second accommodation portion 320B, and the second opening portion 340B may be fluidly connected to each other.

The cartridge 300 may include a partition wall 325 that separates the first accommodation portion 320A and the second accommodation portion 320B from each other. The partition wall 325 may be arranged between the first accommodation portion 320A and the second accommodation portion 320B. A target substance may be stored in each of the first accommodation portion 320A and the second accommodation portion 320B divided by the partition wall 325.

The partition wall 325 may provide an inclined surface to the first accommodation portion 320A and the second accommodation portion 320B. For example, a second surface 322A of the first accommodation portion 320A may correspond to one surface of the partition wall 325, and a second surface 322B of the second accommodation portion 320B may correspond to the other surface of the partition wall 325 facing the opposite direction to one surface of the partition wall 325.

According to this structure, while maintaining the concept of arranging the inclined surface in the accommodation portion 320, the capacity of the cartridge 300 capable of storing the target substance may be increased. In addition, because two accommodation portions, that is, the first accommodation portion 320A and the second accommodation portion 320B, are separated by the partition wall 325, different types of target substances may be stored in the two accommodation portions, respectively. In this case, the user may inhale a target substance of a desired type by turning the cartridge 300 upside down and mounting the cartridge 300 on the main body 100 at any time.

As described above, because the passage portion 330 has a shape that is bent at least once, it may be difficult for the target substance stored in the accommodation portion 320 to leak out through the two passage portions 330 regardless of a direction in which the cartridge 300 is mounted.

However, because the two opening portions 340 are respectively open to upper and lower parts of the housing 310, a problem in which the target substance leaks out through the opening portions 340 may occur. For example, as illustrated in FIG. 9A, when the first opening portion 340A is positioned at the upper part of the housing 310 and connected to the flow path 130 of the main body 100, the second opening portion 340B is positioned at the lower part of the housing 310, and thus, the target substance stored in the second accommodation portion 320B may spill out through the second opening 340B.

Hereinafter, a configuration arranged to solve the problem of target substance leaking through the opening portion 340 will be described.

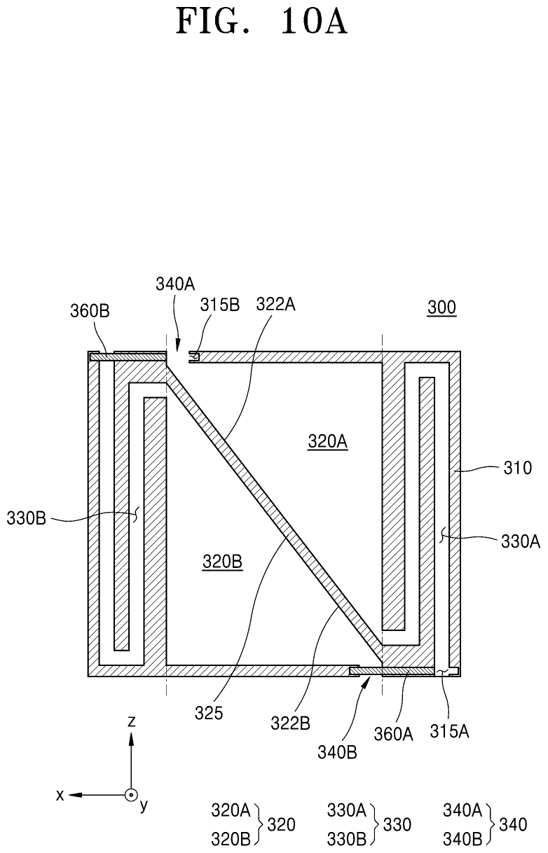

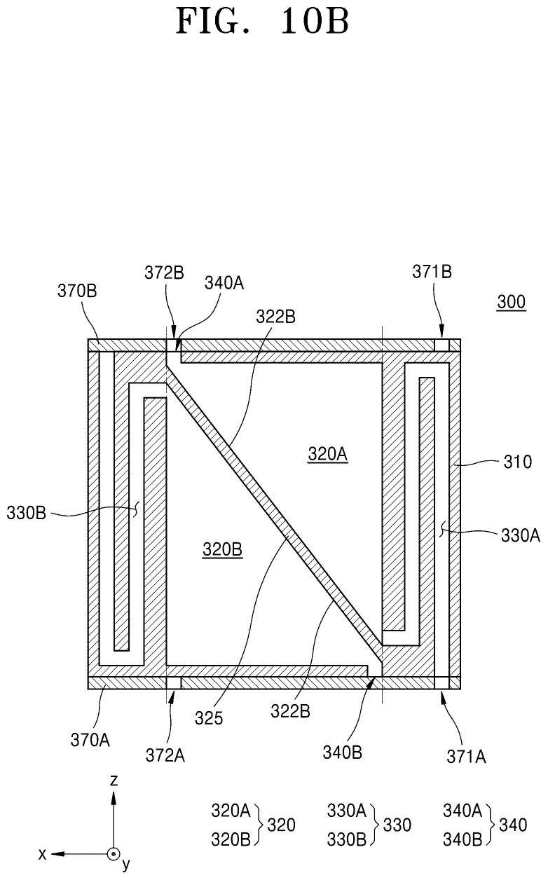

FIG. 10A is a cross-sectional view of a cartridge according to another embodiment in which a door is applied to the cartridge of FIG. 9A. FIG. 10B is a cross-sectional view of a cartridge according to another embodiment in which a cartridge blocking portion of FIG. 9A is applied.

Cartridges 300 illustrated in FIGS. 10A and 10B correspond to separate embodiments for solving the problem of target substance leaking through the opening portion 340.

At least one of the components of the cartridges 300 illustrated in FIGS. 10A and 10B may be identical or similar to at least one of the components of the cartridges 300 illustrated in FIGS. 9A and 9B, and any duplicate description is omitted below.

Referring to FIG. 10A, the cartridge 300 illustrated in FIG. 10A may further include doors 360A and 360B compared to the cartridge 300 illustrated in FIG. 9A.

The doors 360A and 360B are configured to slide to open and close the passage portion 330 or the opening portion 340. At least parts of the doors 360A and 360B may be respectively accommodated in sliding grooves 315A and 315B formed in the housing 310. The doors 360A and 360B and the sliding grooves 315A and 315B are similar to the door 260 and the sliding groove 215 illustrated in FIG. 6, respectively, and thus, any duplicate description is omitted below.

Two doors 360A and 360B may be arranged. Specifically, the doors 360A and 360B may include a first door 360A that opens and closes the first passage portion 330A or the second opening portion 340B, and a second door 360B that opens and closes the second passage portion 330B or the first opening portion 340A. The first door 360A and the second door 360B may move independently.

In order to respond to the two doors 360A and 360B, two sliding grooves 315A and 315B may also be formed in the housing 310. That is, the sliding grooves 315A and 315B may include a first sliding groove 315A that accommodates the first door 360A and a second sliding groove 315B that accommodates the second door 360B.

Referring to the drawings, the cartridge 300 is arranged in a direction in which a target substance stored in the first accommodation portion 320A may be inhaled. Accordingly, a target substance stored in the first accommodation portion 320A may move to the main body through the first opening portion 340A, and a target substance stored in the second accommodation portion 320B may flow out of the inhaler through the second opening portion 340B.

In order to prevent the target substance from flowing out through the second opening portion 340B, the second opening portion 340B has to be closed. To this end, the first door 360A may move along the first sliding groove 315A to close the second opening portion 340B. At the same time, the first door 360A may open the first passage portion 330A so that the user may use the inhaler.

Because the first opening portion 340A has to be opened for the inhalation of the target substance, the second door 360B may move along the second sliding groove 315B to open the first opening portion 340A. At the same time, in order to prevent the target substance stored in the second accommodation portion 320B from flowing out through the second passage portion 330B and to prevent foreign materials from flowing in through the second passage portion 330B, the second door 360B may close the second passage portion 330B. However, because the air-introducing inlet of the second passage portion 330B is arranged to face the main body of the inhaler, the air-introducing inlet of the second passage portion 330B is covered by the main body and is not exposed to the outside of the inhaler. Therefore, in this case, the importance of closing the second passage portion 330B may be somewhat reduced.

As shown in the drawings, when the doors 360A and 360B close the opening portion 340, the passage portion 330 may be opened, and when the doors 360A and 360B close the passage portion 330, the opening portion 340 may be opened. However, embodiments are not limited to this opening/closing method.

As another example, the doors 360A and 360B may simultaneously open/close the opening portion 340 and the passage portion 330. As another example, the doors 360A and 360B may be respectively arranged in the opening portion 340 and the passage portion 330, and thus, the doors 360A and 360B may independently open/close the opening portion 340 and the passage portion 330, respectively.

Referring to FIG. 10B, the cartridge 300 illustrated in FIG. 10B may further include blocking portions 370A and 370B, compared to the cartridge 300 illustrated in FIG. 9A.

The blocking portions 370A and 370B are configured to be detachably coupled to a part of the housing 310 to open and close the passage portion 330 or the opening portion 340. The blocking portions 370A and 370B may be coupled to the housing 310 by using a rail portion (not shown) arranged in the housing 310. The blocking portions 370A and 370B and the rail portion are similar to the blocking portion 270 and the rail portion illustrated in FIG. 8, respectively, and thus, any duplicate description is omitted.

Two blocking portions 370A and 370B may be arranged. Specifically, the blocking portions 370A and 370B may include a first blocking portion 370A that opens and closes the first passage portion 330A or the second opening portion 340B, and a second blocking portion 370B that opens and closes the second passage portion 330B or the first opening portion 340A. In order to respond to the two blocking portions 370A and 370B, two rail portions may also be arranged.

The blocking portions 370A and 370B may be coupled to the housing 310 even when the blocking portions 370A and 370B are in a state of left-right symmetry (e.g., rotated 180 degrees around the z-axis) or flipped over by rotating 180 degrees around the y-axis in the illustrated state. Accordingly, the user may couple the blocking portions 370A and 370B to the housing 310 without distinguishing between the lefts and the rights of or the tops and the bottoms of the blocking portions 370A and 370B.

The blocking portion 370A may include an inlet opening 371A connected to the passage portion 330 and an outlet opening 372A connected to the opening portion 340, and the blocking portion 370B may include an inlet opening 371B connected to the passage portion 330 and an outlet opening 372B connected to the opening portion 340. However, when the blocking portions 370A and 370B are coupled to the housing 310, the inlet opening portions 371A and 371B and the outlet openings 372A and 372B may not be simultaneously connected to the passage portion 330 and the opening portion 340, respectively.

For example, when the inlet openings 371A and 371B of the blocking portions 370A and 370B are connected to the passage portion 330, the outlet openings 372A and 372B may not be connected to the opening portion 340. In this case, when the user rotates the blocking portions 370A and 370B 180 degrees around the y-axis to flip the blocking portions 370A and 370B upside down and couples the blocking portions 370A and 370B to the housing 310, the outlet openings 372A and 372B may be connected to the opening portion 340, but the inlet openings 371A and 371B may not be connected to the passage portion 330.

Accordingly, when the user wants to close the second opening portion 340B located at the bottom of the housing 310, the user may couple the first blocking portion 370A to the housing 310 as illustrated. In this case, the first blocking portion 370A may close the second opening portion 340B. At the same time, the first passage portion 330A may be opened by being connected to the first inlet opening 371A so that the user may use the inhaler. In this case, the first outlet opening 372A may be located at a location unrelated to the location of the second opening portion 340B.

Because the first opening portion 340A located at the upper part of the housing 310 has to be open, the user may couple the second blocking portion 370B to the housing 310 as illustrated.

In this case, the first opening portion 340A may be opened by being connected to the second outlet opening 372B. At the same time, the second passage portion 330B may be closed by the second blocking portion 370B. In this case, the second inlet opening 372B may be located at a location unrelated to the location of the second passage portion 330B.

Hereinafter, an example of a method of coupling a cartridge to a main body will be described.

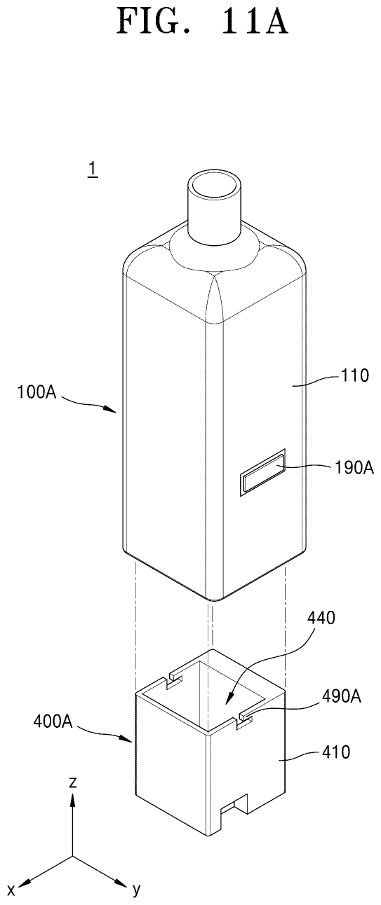

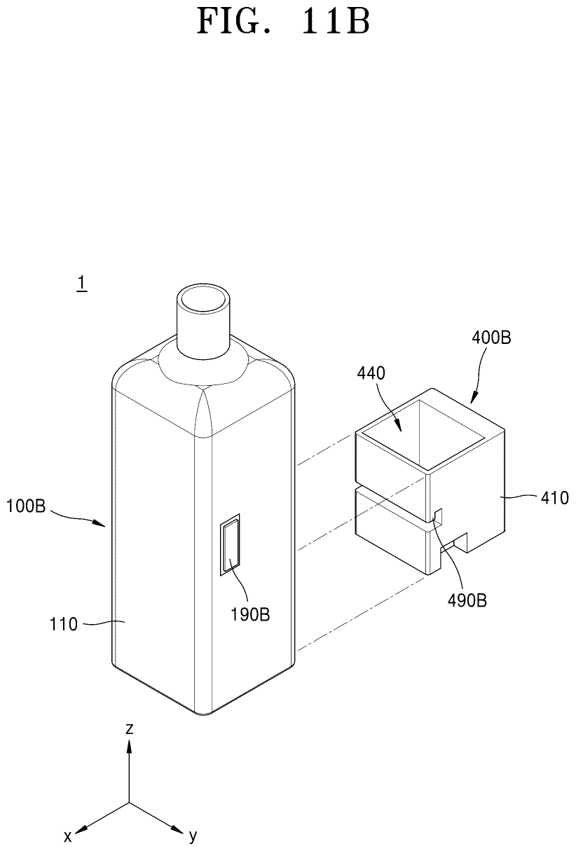

FIGS. 11A and 11B are perspective views showing a main body of an inhaler and a cartridge separated therefrom, according to embodiments to which a fastening structure is applied.

Referring to FIGS. 11A and 11B, an inhaler 1 according to another embodiment may include a main body 100 and a cartridge 400A or 400B.

Referring to FIG. 11A, an embodiment, in which a cartridge 400A approaches a lower part of the main body 100 as described above and is inserted into the main body 100 in a length direction of the main body 100 (e.g., in the z-axis direction), is illustrated.

Referring to FIG. 11B, an embodiment, in which a cartridge 400B approaches a side portion of the main body 100 and is inserted into the main body 100 in a direction crossing the length direction of the main body 100 (e.g., in the x-axis direction), is illustrated.

The main body 100 of the inhaler 1 of FIG. 11A may include an operating portion 190A, and the main body 100 of the inhaler 1 of FIG. 11B may include an operating portion 190B. The operating portions 190A and 190B are used to detachably couple the cartridges 400A and 400B to the main body 100, respectively. In this case, only buttons among the sub-components of the operating portions 190A and 190B may be exposed on the outside of the main body 100.

The cartridges 400A and 400B may be engaged with parts of the operating portions 190A and 190B, respectively. To this end, a fastening groove may be formed in an area of the housing 410 of each of the cartridges 400A and 400B, and step portions 490A and 490B may be respectively arranged in areas where the fastening groove is formed.

Each of the step portions 490A and 490B may protrude inside the fastening groove in a direction that crosses a direction in which a groove is dug in the housing 410. In this case, the step portions 490A and 490B may be components included in the housing 410 or may be sub-components of the cartridges 400A and 400B that are handled separately from the housing 410.

Referring to FIG. 11A, the fastening groove and the step portion 490A are arranged on an upper part of the cartridge 400A that approaches a lower part of the main body 100. In this case, considering the arrangement of an opening portion 440, the fastening groove and the step portion 490A may be arranged on both sides of the opening portion 440, respectively.

Referring to FIG. 11B, the fastening groove and the step portion 490B are arranged on a side part of the cartridge 400B that approaches a side part of the main body 100. As shown in the drawings, the fastening groove is formed to cross one side of the side part of the cartridge 400B, but embodiments are not necessarily limited thereto.

Hereinafter, a fastening structure of the main body 100 and the cartridge 400B will be described in detail based on the embodiment illustrated in FIG. 11B.

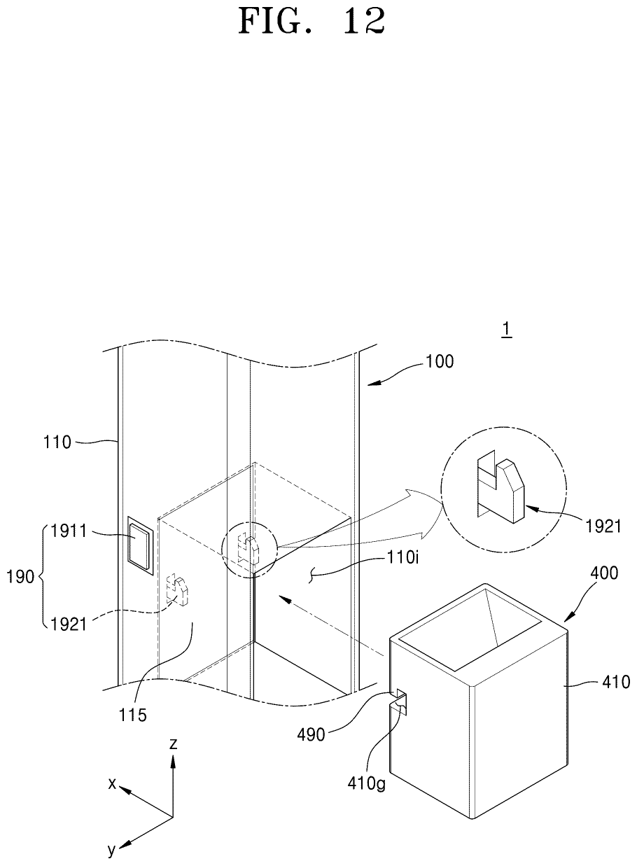

FIG. 12 is a perspective view of the main body and cartridge illustrated in FIG. 11B, viewed from a different angle to explain the internal structure of the main body of an inhaler.

Referring to FIG. 12, an inhaler 1 according to another embodiment may include a main body 100 and a cartridge 400.

At least one of the components of the inhaler 1 illustrated in FIG. 12 may be identical or similar to at least one of the components of the inhaler 1 illustrated in FIG. 11B, and any duplicate description is omitted below.

Referring to FIG. 12, the cartridge 400 may be inserted into the interior of the main body 100 through the side of the main body 100. In this case, the interior of the accommodation space 110i of the body 110 is illustrated in FIG. 12.

As illustrated in FIG. 12, at least a part of an operating portion 190 is not illustrated because the at least a part of the operating portion 190 is covered by a cover plate 115. In this case, the cover plate 115 may correspond to a component of the body 110, or may be a sub-component of the main body 100 that is handled separately from the body 110.

When only the configuration of the operating portion 190 illustrated in FIG. 12 is described, the operating portion 190 may include a button 1911 and a fastening member 1921.

The button 1911 may be positioned so that at least a part thereof is exposed to the outside of the body 110, and may perform a function of releasing the fastening relationship between the main body 100 and the cartridge 400 according to a user's input. For example, when a user's input is applied to the button 1911, the cartridge 400 may be detached from the main body 100.

The fastening member 1921 is configured to be engaged with a step portion 490 of the cartridge 400 and seated in a fastening groove 410g. As the fastening member 1921 moves up and down, the cartridge 400 may be fastened to the main body 100 or may be detachable from the main body 100.

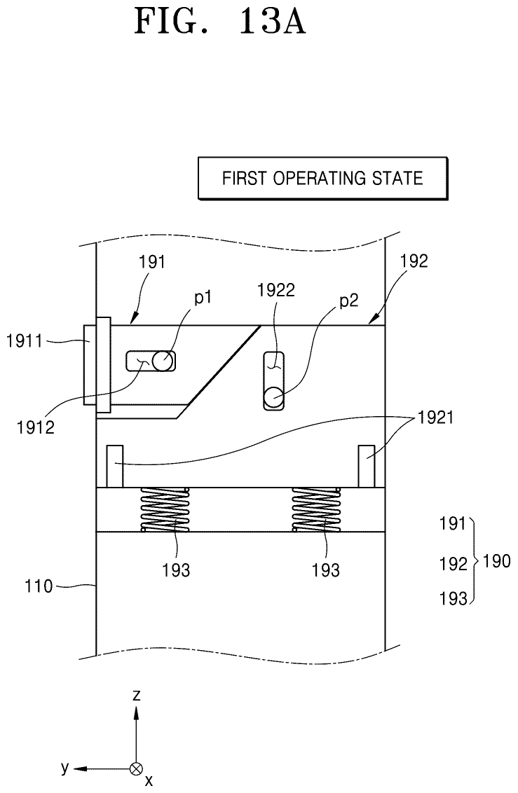

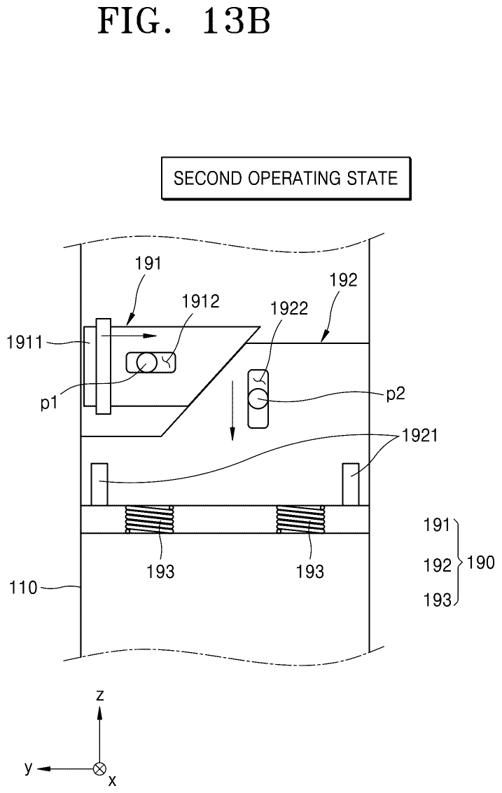

FIG. 13A is an exploded front view showing the inhaler main body illustrated in FIG. 12 in a first operating state. FIG. 13B is an exploded front view showing the inhaler main body illustrated in FIG. 12 in a second operating state.

In this case, the ‘first operating state’ refers to a state in which no input is applied to the button 1911 and thus the cartridge (e.g., the cartridge 400 of FIG. 12) is mounted on the main body 100, and the ‘second operating state’ refers to a state in which a user's input is applied to the button 1911 and thus the cartridge is allowed to be removed from the main body 100. The above expressions may be used with the same meaning below.

Referring to FIGS. 13A and 13B, the main body 100 of the inhaler 1 according to another embodiment may include a body 110 and an operating portion 190. In this case, the operating portion 190 may include a first moving member 191, a second moving member 192, and an elastic member 193.

The first moving member 191 is configured to be slidable in a horizontal direction according to a user's operation. The first moving member 191 may include a button 1911 and a first guide hole 1912.

The button 1911 is arranged so that at least a part thereof is exposed to the outside of the body 110, and may be arranged at one end of the first moving member 191. When the user presses the button 1911 to move the button 1911, the first moving member 191 may also move.

The first guide hole 1912 is formed in one area of the first moving member 191 and extends in a horizontal direction, which is the moving direction of the first moving member 191. A first guide protrusion P1 may be inserted into the first guide hole 1912. The first guide protrusion P1 is arranged inside the body 110 and is configured to be fixed at one position.

Because the first guide protrusion P1 is inserted into the first guide hole 1912 extending in the horizontal direction, the first moving member 191 may move only in the horizontal direction. In addition, because the first guide protrusion P1 comes into contact with a part of the first moving member 191 at both ends of the first guide hole 1912, the horizontal movement of the first moving member 191 may be restricted.

The second moving member 192 is configured to be slidable in a vertical direction by the sliding movement of the first moving member 191 by engaging with the first moving member 191. The second moving member 192 may include a fastening member 1921 and a second guide hole 1922.

The fastening member 1921 is positioned to be exposed to the accommodation space (e.g., the accommodation space 110i of FIG. 12) as shown in FIG. 12, and may move together with the second moving member 192.

The second guide hole 1922 is formed in one area of the second moving member 192 and extends in a vertical direction, which is the moving direction of the second moving member 192. A second guide protrusion P2 may be inserted into the second guide hole 1922. The second guide protrusion P2 is positioned inside the body 110 and is configured to be fixed at one position.

Because the second guide protrusion P2 is inserted into the second guide hole 1922 extending in the vertical direction, the second moving member 192 may move only in the vertical direction. In addition, because the second guide protrusion P2 comes into contact with a part of the second moving member 192 at both ends of the second guide hole 1922, the vertical movement of the second moving member 192 may be restricted.

A part of the surface where the first moving member 191 and the second moving member 192 come into contact with each other may be an inclined surface. Therefore, the second moving member 192 may move in the vertical direction by a force applied by the first moving member 191 through the inclined surface. Conversely, the first moving member 191 may also move in the horizontal direction by a force applied by the second moving member 192 through the inclined surface.

The elastic member 193 is configured to elastically press the second moving member 192 in a direction (e.g., vertically upward) toward the first moving member 191. When the second moving member 192 moves in a vertically downward direction due to the pressing of the first moving member 191, the elastic member 193 may be compressed.

The compressed elastic member 193 may apply an elastic force to the second moving member 192 to restore the second moving member 192 to a position when there is no pressing. Accordingly, the first moving member 191 may also move to a position when there is no pressing.

Hereinafter, the movement of the first moving member 191 and the second moving member 192 based on the user's input to the button 1911 will be described in detail.

Referring to FIG. 13A, the main body 100 in the first operating state is illustrated. In a state where there is no pressing, the first moving member 191 is positioned so that the button 1911 is exposed to the outside of the body 110. In this case, the first moving member 191 may be moved to the left as much as possible. The second moving member 192 may also be moved upward as much as possible. Accordingly, the fastening member 1921 may also be moved upward as much as possible.

Referring to FIG. 13B, the main body 100 in the second operating state is illustrated. When the user presses the button 1911, the first moving member 191 may move horizontally in the inward direction of the main body 100. As a result, the second moving member 192 may move vertically in the downward direction of the main body 100, and the elastic member 193 in contact with a lower part of the second moving member 192 may be compressed. In this case, the first moving member 191 may be moved to the right as much as possible. The second moving member 192 may also be moved downward as much as possible. Accordingly, the fastening member 1921 may also be moved downward as much as possible.

When the user presses the button 1911 so that the fastening member 1921 moves downward, the cartridge 400 may be separated from the main body 100. After the cartridge 400 is separated, when the pressure on the button 1911 is released, the internal components of the main body 100 may be restored to the first operating state by the elastic force of the elastic member 193.

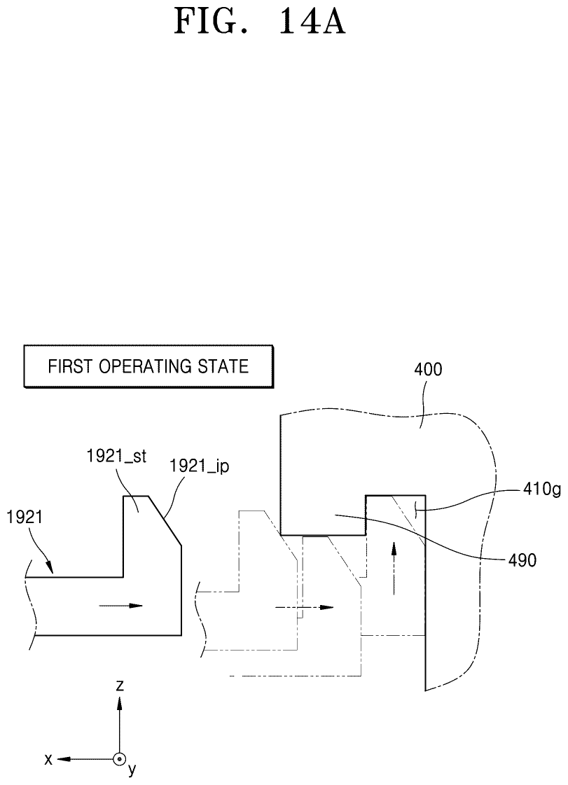

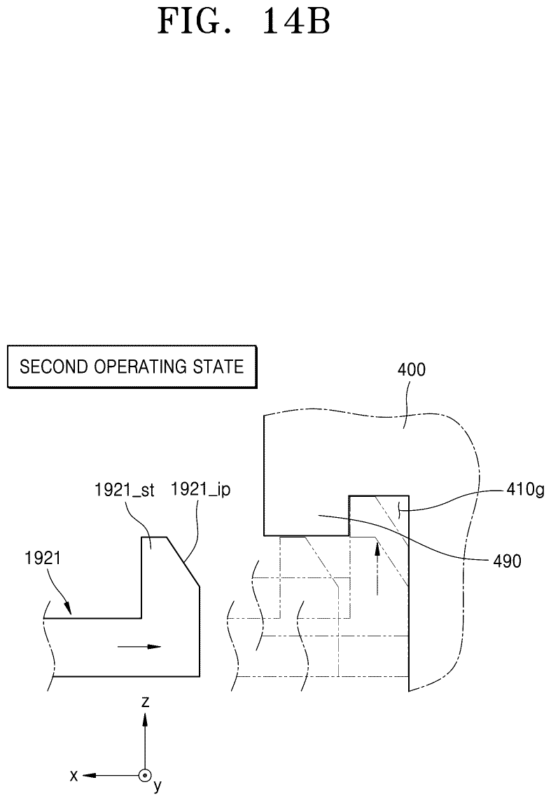

FIG. 14A is a cut-away side view schematically showing a state where a fastening groove of a cartridge is coupled to an inhaler main body through fastening with a fastening member in the first operating state. FIG. 14B is a cut-away side view schematically showing a state where a fastening groove of a cartridge is coupled to an inhaler main body through fastening with a fastening member in the second operating state.

Hereinafter, when describing FIGS. 14A and 14B, reference will be made to the drawing symbols of FIG. 12.

Referring to FIGS. 14A and 14B, a fastening member 1921 may include a stopper 1921_st that protrudes upward. A fastening groove 410g may be formed to be drawn upward to accommodate the stopper 1921_st.

When in the first operating state, the stopper 1921_st may be accommodated in the fastening groove 410g to limit movement of the cartridge 400 in the x-axis direction. In addition, when in the second operating state, the stopper 1921_st may move downward and be separated from the fastening groove 410g to allow movement of the cartridge 400 in the x-axis direction.

Specifically, the stopper 1921_st may include an inclined portion 1921_ip. In order to mount the cartridge 400 on the main body 100 in a state (e.g., in the first operating state) where the user does not make any input to the button 1911, when the cartridge 400 is pushed toward the main body 100 at a height where the fastening groove 410g and the stopper 1921_st correspond to each other, the outer edge of the step portion 490 of the cartridge 400 and the inclined portion 1921_ip of the stopper 1921_st may come into contact with each other, as shown in FIG. 9A.

In this state, when the cartridge 400 is pushed further toward the main body 100, the outer edge of the step portion 490 slides against the inclined portion 1921_ip, thereby moving the stopper 1921_st downward. In this case, the stopper 1921_st may move downward until the upper surface of the stopper 1921_st comes into contact with the lower surface of the step portion 490.

When the cartridge 400 continues to be pushed toward the main body 100 while the upper surface of the stopper 1921_st comes into contact with the lower surface of the step portion 490, the stopper 1921_st may reach the fastening groove 410g.

Then, the upper surface of the stopper 1921_st no longer comes into contact with the lower surface of the step portion 490. The stopper 1921_st may move upward from the fastening groove 410g and be fitted into the fastening groove 410g. In this case, the position of the stopper 1921_st may be the same as the position of the stopper 1921_st before moving downward.

Through the above-described process, the fastening member 1921 is accommodated in the fastening groove 410g so that the cartridge 400 may be mounted on the main body 100.

According to the structure described above, the user may mount the cartridge 400 on the main body 100 without applying an input to the button 1911.

Furthermore, as shown in FIG. 9B, in a state (e.g., the second operating state) where the user applies an input to the button 1911, the cartridge 400 may be easily mounted on the main body 100 without contact between the inclined portion 1921_ip of the stopper 1921_st and the cartridge 400.

However, when attempting to separate the cartridge 400 from the main body 100, the user has to press the button 1911 to move the stopper 1921_st downward.

In summary, mounting the cartridge 400 on the main body 100 is possible in the first operating state and also in the second operating state, but the cartridge 400 may be more easily mounted on the main body 100 in the second operating state. Detaching the cartridge 400 from the main body 100 is possible only in the second operating state.

That is, in order to maintain the state in which the cartridge 400 is mounted on the main body 100, the inhaler has to maintain the first operating state in which the movement of the cartridge 400 in the x-axis direction is restricted.

According to a cartridge according to embodiments and an inhaler including the cartridge, because an accommodation portion where a target substance is stored has an inclined surface, air may easily transport the target substance, and thus, the target substance may be effectively supplied to the user.

In addition, according to a cartridge according to embodiments and an inhaler including the cartridge, a target substance may be prevented from leaking through a portion other than a mouthpiece, due to the complex shape (e.g., a shape bent at least once) of a passage portion.

In addition, according to a cartridge according to embodiments and an inhaler including the cartridge, particles larger than a certain size may be prevented from entering the user by using a cyclone structure, and the inhalation sensation of the target substance may be improved.

Effects of the present disclosure are not limited to the above effects, and effects that are not mentioned could be clearly understood by one of ordinary skill in the art from the present specification and the attached drawings.

Certain embodiments or other embodiments of the present disclosure described above are not exclusive or distinct from each other. The certain embodiments or other embodiments of the present disclosure described above may be combined with each other or used in combination with each other in their respective components or functions.

For example, it means that an A component described in a specific embodiment and/or the drawings and a B component described in another embodiment and/or the drawings may be combined with each other. In other words, even when it is not explained directly about combination between components, it is possible to combine unless it is explained that combination is impossible.

The above detailed description should not be interpreted restrictedly but should be considered illustrative in all aspects. The scope of the present disclosure should be determined by a rational interpretation of the attached claims, and all changes within the equivalent scope of the present disclosure are included in the scope of the present disclosure.

What is claimed is:

1. A cartridge for an inhaler, comprising:
a housing forming an exterior of the cartridge;
an accommodation portion formed in the housing to accommodate a target substance;
a passage portion formed in the housing to fluidly connect an exterior of the housing to the accommodation portion; and
an opening portion formed in the housing to allow the target substance accommodated in the accommodation portion to pass therethrough,
wherein the accommodation portion comprises a first surface extending in a first direction and a second surface inclined with respect to the first direction,
one end of the second surface is connected to the passage portion, and
another end of the second surface is connected to the opening portion.

2. The cartridge of claim 1, wherein the passage portion comprises: an air-introducing inlet connected to the exterior of the housing; and an air inlet connected to the accommodation portion,
wherein the opening portion is open in the first direction, and the air inlet is open in a second direction crossing the first direction.

3. The cartridge of claim 1, wherein the passage portion comprises:
an air-introducing inlet open toward the outside of the housing;
a first passage extending from the air-introducing inlet in the first direction;
a second passage connected to the first passage and extending in a second direction crossing the first direction;
a third passage connected to the second passage and extending in the first direction; and
an air inlet connected to the third passage and open toward the accommodation portion.

4. The cartridge of claim 3, wherein a cross-sectional area of a cross-section of the first passage increases away from the air-introducing inlet, the cross-section being taken in the second direction.

5. The cartridge of claim 3, wherein the accommodation portion and the passage portion are arranged parallel in the second direction, which is a direction crossing the first surface, and the first passage and the third passage of the passage portion are arranged parallel in the second direction.

6. The cartridge of claim 1, further comprising a blocking portion detachably coupled to the housing and configured to block at least a part of the opening portion,
wherein the blocking portion comprises an air outlet fluidly connecting the outside of the housing to the accommodation portion.

7. The cartridge of claim 1, wherein the opening portion is arranged to face the first surface.

8. The cartridge of claim 1, wherein the accommodation portion comprises a first accommodation portion and a second accommodation portion, the passage portion comprises a first passage portion and a second passage portion, the opening portion comprises a first opening portion and a second opening portion, the first passage portion, the first accommodation portion, and the first opening portion are fluidly connected to each other, and the second passage portion, the second accommodation portion, and the second opening portion are fluidly connected to each other,
the cartridge further comprising a partition wall separating the first accommodation portion from the second accommodation portion,
wherein the second surface of the first accommodation portion is one surface of the partition wall, and the second surface of the second accommodation portion is another surface of the partition wall facing in an opposite direction to the one surface of the partition wall.

9. The cartridge of claim 1, further comprising a door configured to open and close the passage portion.

10. The cartridge of claim 1, further comprising a groove formed by a recess in an edge of the housing and one surface of the housing comprising the edge,
wherein the groove comprises a first groove arranged on one surface of the housing and a second groove arranged on another surface of the housing opposite to the one surface.

11. An inhaler comprising:
a cartridge comprising a housing, an accommodation portion formed in the housing to accommodate a target substance, a passage portion formed in the housing to fluidly connect an exterior of the housing to the accommodation portion, and an opening portion formed in the housing to allow the target substance accommodated in the accommodation portion to pass therethrough; and
a main body to which the cartridge is detachably coupled, the main body comprising a body including an accommodation space for accommodating the cartridge, a mouthpiece protruding out of the body and configured to contact a user's mouth, and a flow path connecting the opening portion of the cartridge to the mouthpiece,
wherein the accommodation portion comprises a first surface extending in a first direction and a second surface inclined with respect to the first direction, one end of the second surface is connected to the passage portion, and another end of the second surface is connected to the opening portion.

12. The inhaler of claim 11, wherein the flow path comprises:
an inlet portion open toward the accommodation space of the body;
a chamber arranged below the mouthpiece and having a cylindrical shape;
a connection portion extending from an end of the inlet portion in a tangential direction of the cylindrical shape and connected to the chamber; and
an outlet portion extending from the chamber to the mouthpiece.

13. The inhaler of claim 12, wherein a cross-sectional area of a cross-section of the inlet portion decreases toward the connection portion, the cross-section being taken in a direction crossing a direction in which the inlet portion extends.

14. The inhaler of claim 12, further comprising a protrusion comprising a hollow connected to the mouthpiece, the protrusion protruding inwardly from the body,
wherein the chamber accommodates the protrusion, and the outlet portion extends to the mouthpiece along the hollow of the protrusion.

15. The inhaler of claim 14, further comprising a guide portion coupled to an outer surface of the protrusion and comprising a plurality of wings configured to guide movement of air so that the air rotates inside the chamber,
wherein the chamber comprises a first region having a cylindrical shape in which the guide portion is arranged and a second region in which an end of the protrusion is arranged, and the second region becomes narrower away from the first region.
